(19) United States
(12) Patent Application Publication
(10) Pub. No.: US 2010/0297224 A1
(43) Pub. Date: Nov. 25, 2010

(54) NSAID Dose Unit Formulations with H2-Receptor Antagonists and Methods of Use

(75) Inventors: George Tidmarsh, Portola Valley, CA (US); Barry L. Golombik, Incline Village, NV (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Horizon Therapeutics, Inc., Palo Alto, CA (US)

(21) Appl. No.: 12/438,701

(22) PCT Filed: Aug. 29, 2007
(86) PCT No.: PCT/US07/77106

§ 371 Date: Aug. 10, 2010

Related U.S. Application Data
(60) Provisional application No. 60/824,264, filed on Aug. 31, 2006.

(51) Int. Cl. A61K 31/426; A61K 9/10; A61K 9/22; A61K 9/52; A61P 29/00

(52) U.S. Cl. 424/457; 514/370; 424/484; 424/468

(57) ABSTRACT

The present invention generally relates to pharmaceutical unit dosage forms of NSAIDs and H2-receptor antagonists, in which the H₂-receptor antagonist is formulated so as to be released in a sustained manner over a predetermined period of time so as to maintain gastric pH above a desired level for a duration of time. The NSAID may then be formulated for immediate release. The pharmaceutical unit dosage forms may be administered to subjects susceptible to the development of NSAID induced gastric and/or duodenal ulcers, as the sustained release H₂-receptor antagonist is formulated so as to maintain the gastric environment above the pH levels where NSAID-induced ulceration typically occurs.

### CROSS-REFERENCE TO RELATED APPLICATIONS

This application claims priority to U.S. provisional application No. 60/824264, tiled Aug. 31, 2006, the entire content of which is incorporated herein by reference.

### BACKGROUND OF THE INVENTION

Non-steroidal anti-inflammatory drugs (“NSAID(s)”) are known to be effective analgesics for the treatment of mild to moderate pain. While generally regarded as safe, NSAIDs can cause gastritis, dyspepsia, and gastric and duodenal ulceration. Gastric and duodenal ulceration is a consequence of impaired mucosal integrity resulting from NSAID-mediated inhibition of prostaglandin synthesis. This side-effect is a particular problem for individuals who take NSAIDs for extended periods of time, such as patients suffering from rheumatoid arthritis and osteoarthritis.

Histamine receptor blockers (referred to generically herein as H₁ or H₂ blockers) are effective inhibitors of gastric acid production. In addition, proton pump inhibitors are known as effective gastric acid inhibitors. The risk of developing gastric or duodenal ulceration can be reduced by limited cotherapy with the drug. famotidine. Famotidine blocks the action of the histamine type-2 (H₂) receptor, leading to a reduction of acid secretion in the stomach. Reducing stomach acid with famotidine during treatment with certain NSAID drugs is reported to decrease incidence of gastrointestinal ulcers (see, e.g., Taha et al., 1996, “Famotidine for the prevention of gastric and duodenal ulcers caused by nonsteroidal anti-inflammatory drugs” N Engl J Med 334:1435-9, and Rostom et al., 2002, “Prevention of NSAID-induced gastrointestinal ulcers” Cochrane Database Syst Rev 4:CD002296).

Although NSAID plus famotidine cotherapy reduces risk of developing gastric or duodenal ulceration, present therapies are not widely used. More effective methods of treatment and pharmaceutical compositions are needed. The present invention meets this and other needs.

### BRIEF SUMMARY OF THE INVENTION

In one aspect, the present invention provides a pharmaceutical unit dosage form comprising: (a) a first component comprising an amount of an H₂-receptor antagonist effective to raise gastric pH above about 3.5; and (b) a second component comprising a therapeutically effective amount of an NSAID. The first component is generally formulated so as to allow for sustained release of the H₂-receptor antagonist at the desired effective amount over a predetermined period of time, and the second component is formulated to allow for immediate release of the NSAID.

In certain embodiments, the first component comprises a release modifying agent to, at least in part, provide for said sustained release. Exemplary release modifying agents include polymers selected from the group consisting of: cellulosic materials, polyvinyl acetates, polyvinyl alcohols, polyethylene oxides, polyethylene glycols, metacrylates, non-crosslinked polyvinylpyrolidone, and combinations thereof.

In preferred embodiments, the H₂-receptor antagonist consists essentially of famotidine or a pharmaceutically acceptable salt thereof. In yet other preferred embodiments. the NSAID consists essentially of naproxen, or a pharmaceutically acceptably salt thereof.

In other aspects, the present invention relates to a pharmaceutical unit dosage form comprising: (a) a first sustained release component comprising an amount of an H₂-receptor antagonist effective to raise gastric pH above about 3.5 for at least 4 hours, and at least one release modifying agent; and (b) a second immediate release component comprising a therapeutically effective amount of an NSAID; wherein the first sustained release component is formulated as a tablet and the second immediate release component is formulated as a flowable powder; and wherein the pharmaceutical unit dosage form is a capsule comprising said tablet and flowable powder.

In certain embodiments, the H₂-receptor antagonist consists essentially of famotidine or a pharmaceutically acceptable salt thereof; the NSAID consists essentially of naproxen, or a pharmaceutically acceptably salt thereof; and the at least one release modifying agent is a polymer selected from the group consisting of: cellulosic materials, polyvinyl acetates, polyvinyl alcohols, polyethylene oxides, polyethylene glycols, metacrylates, non-crosslinked polyvinylpyrolidone, and combinations thereof.

In yet other aspects of the invention, a pharmaceutical unit dosage form is provided which comprises: (a) a sustained release component comprising from about 20 to about 60 mg of famotidine, or a pharmaceutically acceptable salt thereof, and at least one release modifying agent: and (b) an immediate release component comprising from about 200 to about 600 mg of naproxen, or a pharmaceutically acceptable salt thereof.

Yet another aspect of the invention relates to a method for treating osteoarthritis in a subject susceptible to developing NSAID induced gastric and duodenal ulcers. The method comprises administering a pharmaceutical unit dosage form of the invention to a subject in need thereof.

Yet another aspect of the invention relates to a method for treating or preventing pain or inflammation in a subject susceptible to developing NSAID induced gastric and duodenal ulcers. The method comprises administering a pharmaceutical unit dosage form of the invention to a subject in need thereof.

Yet another aspect of the invention relates to a method for preparing a pharmaceutical unit dosage form. The method comprises: (a) preparing a first sustained release matrix core comprising an amount of an H₂-receptor antagonist effective to raise gastric pH above about 3.5 over a predetermined period of time following administration and at least one release modifying agent; (b) preparing an immediate release component comprising a therapeutically effective amount of an NSAID; and (c) combining the matrix core of step (a) and the immediate release blend of step (b) within a unit dosage form.

In certain embodiments, the sustained release matrix is formulated as a tablet. In other embodiments, the immediate release component is formulated as a powder blend. In yet other embodiments, the pharmaceutical unit dose is a capsule, and the method further comprises compressing the sustained release matrix into the form of a tablet; forming the immediate release component as a flowable powder; and loading the sustained release tablet and flowable immediate release powder into the capsule to form the pharmaceutical unit dose.

These and other embodiments of the present invention along with many of its advantages and features are described in more detail in conjunction with the description and claims below.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention as defined by the embodiments.

The present invention generally relates to pharmaceutical unit dosage forms of NSAIDs and H₂-receptor antagonists, in which the H₂-receptor antagonist is formulated so as to be released in a sustained manner over a predetermined period of time so as to maintain gastric pH above a desired level for a duration of time. The NSAID may then be formulated for immediate release. The pharmaceutical unit dosage forms may be administered to subjects susceptible to the development of NSAID induced gastric and/or duodenal ulcers, as the sustained release H₂-receptor antagonist is formulated so as to maintain the gastric environment above the pH levels where NSAID-induced ulceration typically occurs (below a gastric pH of about 3.5).

The pharmaceutical unit dosage forms of the present invention allow for cotherapy of NSAIDs and H₂-receptor antagonists in a simple and effective manner. The sustained release of the HH₂-receptor antagonist provides for desired gastric pH levels over extended periods of time. This allows for the unit dosage form combination tablet to be administered BID or TID to a subject in need of NSAID therapy, without risk of developing NSAID-induced ulcerations. Such administration may be accomplished without the complexities of enteric coatings, controlled release NSAID administration, multiple layer tableting including immediate release H₂-receptor antagonists to initiate the gastric environment, etc.

### I. Definitions

Unless stated otherwise, the following terms and phrases as used herein are intended to have the following meanings:

When tradenames are used herein, applicants intend to independently include the tradename product and the active pharmaceutical ingredient(s) of the tradename product.

The term “treatment” or “treating,” to the extent it relates to a disease or condition includes preventing the disease or condition from occurring where applicable, inhibiting the disease or condition, eliminating the disease or condition, and/or relieving one or more symptoms of the disease or condition.

A “therapeutically effective amount” is an amount of an active ingredient or its pharmaceutically acceptable salt which eliminates, ameliorates, alleviates, or provides relief of the symptoms for which it is administered.

The terms “solid oral dosage form,” “oral dosage form,” “unit close form,” “dosage form for oral administration,” and the like are used interchangably, and refer to a pharmaceutical composition in the form of a tablet, capsule, caplet, gelcap, geltab, pill and the like.

An “excipient,” as used herein, is any component of an oral dosage form that is not an active ingredient. Excipients include binders, lubricants, diluents, disintegrants, coatings, barrier layer components, glidants, and other components. Excipients are known in the art (see HANDBOOK OF PHARMACEUTICAL EXCIPIENTS, FIFTH EDITION, edited by Rowe et al., McGraw Hill). Some excipients serve multiple functions or are so-called high functionality excipients. For example, talc may act as a lubricant, and an anti-adherent, and a glidant. See Pifferi et at., 2005. “Quality and functionality of excipients”Farmaco. 54:1-14; and Zeleznik and Renak, Business Briefing: Pharmagenerics 2004.

### II. Component Compounds of the Invention

In one aspect, component compounds are provided which are useful in preparation of pharmaceutical unit dosage forms of the invention. Generally, the component compounds of the invention include active ingredients including NSAID and H₂-receptor antagonists.

The term “NSAID,” as used herein, refers to any compound acting as a non-steroidal anti-inflammatory agent for the treatment of pain and/or inflammation. The treatment of pain includes all types of pain, including, but is not limited to, chronic pains, such as arthritis pain (e.g., pain associated with osteoarthritis and rheumatoid arthritis), neuropathic pain, and post-operative pain, chronic lower back pain, cluster headaches, herpes neuralgia, phantom limb pain, central pain, dental pain, neuropathic pain, opioid-resistant pain, visceral pain, surgical pain, bone injury pain, pain during labor and delivery, pain resulting from burns (including sunburn), post partum pain, migraine, angina pain, and genitourinary tract-related pain including cystitis, the term also refers to nociceptive pain or nociception.

The MERCK MANUAL, 16th Edition, Merck Research Laboratories (1990) pp 1308-1309 provide well known examples of NSAIDs. The term NSAID includes, but is not limited to, salicylates, indomethacin, flurbiprofen, diclofenac, ketorolac, naproxen, piroxicam, tebufelone, ibuprofen, etodolac, nabumetone, tenidap, alcofenac, antipyrine, aminopyrine, dipyrone, aminopyrone, phenylbutazone, clofezone, oxyphenbutazone, prexazone, apazone, benzydamine, bucolome, cinchopen, clonixin, ditrazol, epirizole, fenoprofen, floctafeninl, flufenamic acid, glaphenine, indoprofen, ketoprofen, meclofenamic acid, mefenamic acid, niflumic acid, phenacetin, salidifamides, sulindac, suprofen, and tolmetin, including pharmaceutically acceptable salts, isomers and derivatives thereof, and combinations thereof. The salicylates may include acetylsalicylic acid, sodium acetylsalicylic acid, calcium acetylsalicylic acid, salicylic acid, and sodium salicylate. In certain preferred embodiments of the invention, the NSAID is naproxen or at least one pharmaceutically acceptable salt thereof.

NSAIDs have been widely used in osteoarthritis therapy for several years. The following references, hereby incorporated by reference, describe various NSAIDs suitable for use in the invention described herein, and processes for their manufacture: U.S. Pat. No. 3,558,690 to Sallmann and Pfister. (assigned to Ciba Geigy), issued 1971; U.S. Pat. No. 3,843,681 (assigned to American Home Products), issued 1974; U.S. Pat. No. 3,766,263 to Godfrey, (assigned to Reckitt and Colman) issued 1973; U.S. Pat. No. 3,845,215 to Godfrey (assigned to Reckitt and Colman) issued 1974; U.S. Pat. No. 3,600,437 to Marshall (assigned to Eli Lilly), issued 1971; U.S. Pat. No. 3,228,831 to Nicholson and Adams, (assigned to Boots Pure Drug), issued 1966; (U.S. Pat. No. 3,385,886 to Nicholson and Adams, (assigned to Boots Pure Drug) issued 1968; U.S. Pat. No. 3,161,654 to Shen, (assigned to Merck & Co.), issued 1964; U.S. Pat. No. 3,904,682 to Fried and Harrison, (assigned to Syntex), issued 1975; U.S. Pat. No. 4,009,197 to Fried and Harrison, (assigned to Syntex), issued 1977; U.S. Pat. No. 3,591,584 to Lombardino (assigned to Pfizer) issued 1971; U.S. Pat. No. 5,068,458 to Dales et al., (assigned to Beecham Group, PLC.), issued Nov. 26, 1991; U.S. Pat. No. 5,008,283 to Blackburn et al. (assigned to Pfizer, Inc.), issued Apr. 16, 1991; and U.S. Pat. No. 5,006,547 to Loose (assigned to Pfizer), issued Apr. 9, 1991. All of the above patents are hereby incorporated by reference.

The terms “H2 histamine receptor antagonist,” “H₂-receptor antagonist,” and “H₂ antagonist” are used interchangeably and refer to compounds capable of blocking the action of histamine on parietal cells in the stomach, decreasing acid production by these cells. The term specifically includes, without limitation, cimetidine (TAGAMET®), famotidine (PEPCID®), nizatidine (ACCID®) and ranitidine (ZANTAC®), as well as their pharmaceutically acceptable salts, various crystal forms, and prodrug forms.

More particularly, in certain embodiments, H₂-receptor antagonists useful in the pharmaceutical unit dosage forms of the invention may include, but are not limited to: ranitidine, cimetidine, nizatidine, famotidine, pharmaceutically acceptable salts, isomers and derivatives thereof, single enantiomers thereof and combinations thereof. In certain preferred embodiments of the invention, the H₂-receptor antagonist is famotidine, or a pharmaceutically acceptable salt thereof.

“Famotidine” is 3-[2-(diaminomethyleneamino)thiazol-4-ylmethylthio]-N-sulfamoylpropionamidine, including the polymorphic forms designated Form A and Form B (see, e.g., U.S. Pat. Nos. 5,128,477 and 5,120,850) and their mixtures, as well as pharmaceutically acceptable salts thereof. Famotidine can be prepared using art-known methods, such as the method described in U.S. Pat. No. 4,283,408. Famotidine properties have been described in the medical literature (see, e.g., Echizen et al., 1991, Clin Pharmacokinet. 21:178-94).

### III. Salts and Hydrates

Again, any reference to any of the compounds of the invention also includes a reference to a physiologically acceptable salt thereof. Examples of physiologically acceptable salts of the compounds of the invention include salts derived from an appropriate base, such as an alkali metal (for example, sodium), an alkaline earth (for example, magnesium), ammonium and NX₄⁺ (wherein X is C₁-C₄ alkyl). Physiologically acceptable salts of an hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound of an hydroxy group include the anion of said compound in combination with a suitable cation such as Na⁺ and NX₄⁺ (wherein X is independently selected from H or a C₁-C₄ alkyl group).

For therapeutic use, salts of active ingredients of the compounds of the invention will be physiologically acceptable, i.e. they will be salts derived from a physiologically acceptable acid or base. However, salts of acids or bases which are not physiologically acceptable may also find use, for example, in the preparation or purification of a physiologically acceptable compound. All salts, whether or not derived form a physiologically acceptable acid or base, are within the scope of the present invention.

### IV. Pharmaceutical Unit Dosage Formulations

Certain aspects of the invention relate to a pharmaceutical unit dosage form. In certain embodiments, the pharmaceutical unit dosage form includes a first component and a second component.

The first component may generally include an amount of an H₂-receptor antagonist effective to raise gastric pH above about 3.5, above about 4.0, between about 4.5-5.0, etc. In preferred embodiments, the first component is formulated so as to allow for sustained release of the H₂-receptor antagonist at the effective amount over a predetermined period of time such that the gastric pH is maintained above about 3.5 for the predetermined period of time, e.g., over at least about 4 hours, at least about 6 hours, at least about 8 hours, at least about 10 hours, at least about 12 hours, etc.

Any suitable methodology known in the art for providing sustained release of pharmaceutical agents, such as diffusion systems (including reservoir devices and inert polymeric matrices), erodable systems (based on the inherent dissolution of the agent and incorporated excipients), and osmotic systems (drug containing core coated with a semi-permeable membrane having a small orifice) may be used in connection with the present invention. The sustained release of an agent from a pharmaceutical unit dosage form can also be achieved by more than one mechanism. For example, for the same pharmaceutical dosage form, the drug release can occur for example by simultaneous swelling and diffusion, simultaneous diffusion and erosion, and simultaneous swelling, diffusion and erosion.

The H₂-receptor antagonist present in the first component may be directly mixed with pharmaceutical acceptable excipients and/or it may be coated with hydrophilic or hydrophobic agents, which are specifically chosen to regulate the rate of release of the antagonist (e.g., a release modifying agent). Such release modifying agents may be polymeric materials, which are slowly water-soluble and/or slowly gel-forming when exposed to an aqueous medium. Non-limiting examples of such polymeric materials are cellulosic derivatives and modified starches. The composition, quantity, proportions, etc. of the release modifying agent(s) can be varied, depending on the specific requirements and release profile desired.

More particularly, in certain embodiments, the first component may generally be formulated with one or more release modifying agents to, at least in part, provide for the sustained release of the H₂-receptor antagonist. It is understood that modifying the amount, type, composition, etc. of release modifying agent incorporated into the first component will result in an modification in the amount of H₂-receptor antagonist released from the component. In certain embodiments, the first component is formulated so as to provide an essentially stable, linear release from which the H₂-receptor antagonist diffuses at a sustained, steady-state rate for a predetermined duration of time, e.g., over at least about 4 hours, at least about 6 hours, at least about 8 hours, at least about 10 hours, at least about 12 hours, etc. In particular embodiments, the first component is formulated so as to provide zero-order release over the desired time period. If desired, the first component may further include one or more additional excipients, including for example and without limitation, binders, surfactants, diluents, colorants, fillers. disintegrants, glidants, anti-lacking agents, anti-static agents, and combinations thereof, as described in further detail below.

Any suitable release modifying agent known in the art may be used including, but not limited to, polymers such as cellulosic materials, polyvinyl acetates, polyvinyl alcohols, polyethylene oxides, polyethylene glycols, metacrylates, non-crosslinked polyvinylpyrolidone, and combinations thereof. Certain preferred cellulosic materials include hydroxypropyl cellulose, hydroxypropylmethyl cellulose, methyl cellulose, hydroxyethyl cellulose, carboxymethylcellulose, mixtures thereof, and the like. In certain embodiments, hydroxypropylmethyl cellulose (HPMC), sometimes called hypromellose or METHOCEL™, may be used. In certain embodiments, OPADRY™ (available from Colorcon) excipients and/or coatings may be used as release modifying agents, alone or in connection with other suitable release modifying agents. By way of non-limiting example, Opadry II White (Y-22-7719) includes hypromellose (HPMC), polydextrose, polyethylene glycol, titanium dioxide and triacetin.

By way of example, desired amounts of one or more HPMC compounds may be mixed with and/or coated onto the desired amount of H₂-receptor antagonist. The mixture may then be compressed into tablet form if desired, and/or the release modifying agent(s) may be coated onto the tablet. Various methodologies for preparing sustained release compositions suitable for use in connection with the present invention are generally described in U.S. Pat. Nos. 3,870,790; 4,140,755; 4,167,588; 4,226,849; 4,259,314; 4,357,469; 4,369,172; 4,389,393; 4,540,566; 5,009,895, each of which is hereby incorporated by reference in its entirety.

The second component may generally include a therapeutically effective amount of an NSAID. In certain embodiments, the second component is formulated to allow for immediate release of the NSAID.

In certain embodiments, the second component may include other excipients, including for example and without limitation, binders, surfactants, diluents, colorants, fillers, disintegrants, glidants, anti-lacking agents, anti-static agents, and combinations thereof. Suitable diluents include, for example and without limitation, dicalcium phosphate, calcium diphosphate, calcium sulfate, lactose, cellulose, kaolin, mannitol, sodium chloride, starches, powdered sugar, colloidal silicon dioxide. titanium oxide, alumina, talc, colloidal silica, microcrystalline cellulose, mixtures thereof, and the like. Suitable binder materials include, for example and without limitation, starches (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (e.g., hydroxypropyl cellulose, hydroxypropyl methylcellulose, methyl cellulose, hydroxyethyl cellulose, carboxymethylcellulose, Silicified Microcrystalline Cellulose (SMCC) prepared by co-processing microcrystalline cellulose and 2% silicon dioxide, mixtures thereof, and the like). veegum, mixtures thereof, and the like. Suitable lubricants include, for example, magnesium stearate, calcium stearate, stearic acid, mixtures thereof, and the like.

Disintegrants are for example starches, clays, celluloses, alginates, gums, crosslinked polymers, mixtures thereof, and the like. In certain embodiments, croscarmellose sodium may be used. Croscarmellose sodium is a cross linked polymer of carboxymethyl cellulose sodium. Cross linking makes it an insoluble, hydrophilic, highly absorbent material, resulting in swelling properties, and its fibrous nature gives it water wicking capabilities. Croscarmellose sodium may be used in oral pharmaceutical formulations as a disintegrant for capsules, tablets and granules, and may be used in both direct-compression and wet-granulation processes. In certain embodiments, concentrations of up to 5% w/w of croscarmellose sodium may be used.

Suitable surfactants include pharmaceutically acceptable non-ionic, ionic and anionic surfactants. An example of a suitable surfactant is sodium lauryl sulfate. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. If desired, flavoring, coloring and/or sweetening agents may be added as well.

In certain embodiments, the second component may be formulated such that at least about 60%, preferably at least about 75%, more preferably at least about 80%, at least about 90%, at least about 95%, etc., of the weight of the NSAID in the unit dosage form is released within about 20, about 15, or about 10 minutes following administration. Dissolution rates may be determined using the known methods.

The formulations may conveniently be presented in unit dosage form and may be prepared by any of the methods well known in the art of pharmacy. Techniques and formulations generally are found in REMINGTON'S PHARMACEUTICAL SCIENCES (Mack Publishing Co., Easton, Pa.). Such methods include the step of bringing into association the active ingredients (i.e., the first and second components) with any additional excipients. In general the formulations are prepared by uniformly and intimately bringing into association the active ingredient with liquid carriers or finely divided solid carriers or both, and then, if necessary, shaping the product or filling capsules.

Formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. Further, each component (i.e., the first component and second component) may be separated formulated, and then combined into the final pharmaceutical unit dosage form.

In certain embodiments of the invention, the first component may be formulated as a tablet or capsule. The first component tablet may vary in shape and may be, for example, round, ovoid, oblong, cylindrical (e.g., disk shaped) or any other suitable geometric shape, for example rectilinear. Preferably the tablet or capsule has a disk or ovoid shape is shaped like a flattened disk, ovoid or torpedo. The edges of the tablet or capsule may be beveled or rounded. In certain embodiments, the tablet may also be shaped as a caplet (capsule form tablet). Further, the tablets may be scored, embossed or engraved. Further, if desired, the tablet or capsule may be colored. e.g., pink, gray, red, blue. so as to aid in manufacturing and quality control to facilitate combination of the tablet with the second component.

There are three types of general methods of preparation of tablets: (1) dry granulation; (2) direct compression; and (3) wet granulation.

Dry granulation procedures may be utilized where one of the constituents, either the drug or the diluent, has sufficient cohesive properties to be tabletted. This method includes mixing the ingredients, slugging, dry screening, and lubricating, followed by compression.

In direct compression, the powdered material(s) to be included in the solid dosage form is/are compressed directly, without modifying the physical nature of the material itself. Typically, the use of direct compression is limited to those situations where the active ingredient has a requisite crystal structure and physical characteristics required for formation of a pharmaceutically acceptable tablet. For solid dosage forms wherein the drug itself is to be administered in a relatively high dose (e.g., the drug itself constitutes a substantial portion of the total weight of the solid dosage form, such as tablet), the drug itself must exhibit physical characteristics, such as cohesiveness, that make it a good candidate for direct compression with the rest of the ingredients. In other instances, direct compression might be applicable even if the drug itself does not show the desired characteristics, by using excipients which enable direct compression.

The wet granulation procedure includes mixing the powders to be incorporated into a solid dosage form in an appropriate blender (such as a twin shell blender or double-code blender), and then adding solutions of a binding agent to the mixed powders to obtained a granulation. Thereafter, the damp mass is screened (e.g., in a 6-, 8-, 15-, 25-mesh screen), and dried (e.g., by tray drying, using a fluid-bed dryer, a spray dryer, microwave, vacuum, or infra-red dryer).

In certain embodiments, the second component may be formulated as a flowable powder, independently or in combination with a tableted or capsule-based first component. The final pharmaceutical unit dosage form may then optionally be formulated as a capsule comprising the tablet or capsule of the first component and flowable powder the second component. In this regard, the first component, e.g., a tablet or capsule, may preferably be sized and shaped so as to be easily accommodated within a capsule, while allowing for inclusion of the second component within the capsule as well. More particularly, as understood by those skilled in the art, typically tablet and capsule processing may not easily be accommodated within a capsule design. As such, the tablet or capsule configuration of the first component may be specifically sized and shaped so as to be accommodated within the capsule of the unit dosage form taking into account the presence of the second component. Upon administration, the unit dosage form capsule releases the immediate release NSAID flowable powder, and the sustained release tablet or capsule dissolves and releases the H₂-receptor antagonist over time.

In certain embodiments, the pharmaceutical unit dosage forms of the invention are formulated so as to initiate release of the H₂-receptor antagonist and the NSAID (such that release begins to occur) at about the same time. That is, the dosage form is not designed so that one of the ingredients begins to release significantly later than the other.

In certain preferred embodiments. the pharmaceutical unit dosage form may be prepared in any suitable manner, preferable by: (a) preparing a first sustained release matrix core comprising an amount of an H₂-receptor antagonist effective to raise gastric pH above about 3.5 over a predetermined period of time following administration and at least one release modifying agent; (b) preparing an immediate release component comprising a therapeutically effective amount of an NSAID; and (c) combining the matrix core of step (a) and the immediate release blend of step (b) within a unit dosage form. As above. in certain embodiments, the sustained release matrix may be formulated as a tablet. Further, the immediate release component may optionally be formulated as a powder blend, independently or together with the tableted sustained release matrix. In addition, in certain preferred embodiments, the pharmaceutical unit dosage form is a capsule.

In other embodiments, the pharmaceutical unit dosage forms may be prepared by compressing the sustained release matrix into the form of a tablet; forming the immediate release component as a flowable powder; and loading the sustained release tablet and flowable immediate release powder into the capsule to form the pharmaceutical unit dose.

As described supra, the H₂-receptor antagonist may consist essentially of famotidine, or a pharmaceutically acceptable salt thereof. Further, the NSAID may consist essentially of naproxen, or a pharmaceutically acceptably salt thereof. In addition, the release modifying agents of the pharmaceutical unit dosage form may include one or more polymers, including, but not limited to: cellulosic materials, polyvinyl acetates, polyvinyl alcohols, polyethylene oxides, polyethylene glycols, methacrylates, non-crosslinked polyvinylpyrolidone, and combinations thereof. Further, the immediate release component may further include one or more pharmaceutically acceptable excipients selected from the group consisting of cellulose derivatives, cross-linked polymers, sugars, soluble salts, colorants, fillers, disintegrants, glidants, anti-tacking agents and anti-static agents. Again, in certain embodiments, the pharmaceutical acceptable excipients may preferably be colloidal silica, calcium diphosphate, talc, magnesium stearate, and combinations thereof.

The amount of active ingredient included in the various component elements to produce the overall pharmaceutical unit dosage form will vary depending upon the ingredient, the subject to be treated, and the particular disease or condition of interest, as generally recognized by those skilled in the art.

More particularly, in certain embodiments, the pharmaceutical dosage forms of the invention may include from about 200 to about 600 mg per dose. from about 250 to about 500 mg per dose, etc. of naproxen; or one of several NSAIDs from the group of: propionic acid derivatives including ibuprofen (the term ibuprofen is meant to include administration of both the racemic mixture of R- and S-enantiomers and the substantially pure S-enantiomer which is the analgesic active form of ibuprofen) from 200 to 400 mg per dose; fenoprofen from 200 to 600 mg per dose; ketoprofen from 50 to 300 mg per dose, meclofenamate from 50 to 400 mg per dose, mefenamic acid from 250 to 500 mg per dose; piroxicam from 10 to 20 mg per dose; indomethacin from 25 to 200 mg per dose. sulindac from 150 to 400 mg per dose, tolmetin from 200 to 1200 mg per dose; etc. As recognized by those skilled in the art, bulk amounts of the active ingredients may vary depending on the salt form used in the formulation. By way of example, slightly greater amounts may be used, e.g., about 275 to about 550 mg per dose of naproxen if the sodium salt is employed to achieve about 250 to about 500 mg per dose of naproxen.

In certain embodiments, the pharmaceutical dosage form may include from about 20 to about 60 mg per dose, from about 20 to about 30 mg per dose, from about 26 to about 27 mg per dose, etc. of famotidine; or other H₂-receptor antagonists including cimetidine from 150 to 800 mg per dose; ranitidine from 50 to 300 mg per dose; etc.

The dosage ranges described above are preferred adult doses and may vary depending upon the age and weight of the patient as would be known by those skilled in the pharmaceutical arts. Further, the unit dosage forms may be administered BID, TID, etc., as desired. By way of example, the unit dosage forms of the invention may include from about 250 to about 500 mg of naproxen and from about 26 to 27 mg of famotidine, and may be administered TID.

### V. Therapeutic Methods

Another aspect of the invention relates to methods for treating or preventing pain or inflammation in a subject susceptible to developing NSAID induced gastric and duodenal ulcers, treating or preventing osteoarthritis in a subject susceptible to developing. NSAID induced gastric and duodenal ulcers, or have other utilities as described herein, including treatment of any subject in need of NSAID treatment. The methods generally include administering a pharmaceutical unit dosage form to a subject in need thereof.

The pharmaceutical unit dosage forms may preferably be administered BID or TID, depending on the condition, disease or disorder to be treated. As such, the pharmaceutical unit dosage forms may be administered every 8 hours to every 12 hours.

A “subject in need of NSAID treatment” is an individual who receives therapeutic benefit from administration of an NSAID. As generally recognized by those skilled in the art. NSAIDs are generally indicated for treatment of mild to moderate pain, dysmenorrhea, inflammation, osteoarthritis, etc. In one embodiment, the subject in need of NSAID treatment is under treatment for a chronic condition. For example and without limitation, a subject in need of NSAID treatment may be an individual with rheumatoid arthritis, an individual with osteoarthritis, an individual suffering from chronic pain (e.g., chronic low back pain, chronic regional pain syndrome, chronic soft tissue pain), or an individual suffering from a chronic inflammatory condition. In general, a subject under treatment for a chronic condition requires ibuprofen treatment for an extended period, such as at least one month, at least four months, at least six months, or at least one year. In another embodiment, the subject in need of ibuprofen treatment is under treatment for a condition that is not chronic, such as acute pain, dysmenorrhea or acute inflammation. Preferably the patient in need of NSAID treatment does not suffer from a condition characterized by hypersecretion of gastric acid (e.g., Zollinger-Ellison Syndrome). Preferably the patient does not suffer from Barrett's ulceration or active severe oesophagitis. In certain embodiments the subject does not have gastroesophageal reflux disease (GERD). In certain embodiments the subject is not in need of treatment for an ulcer. In certain embodiments the subject does not suffer from dyspepsia. In certain embodiments the subject is susceptible to developing an NSAID-induced ulcer, including gastric and/or duodenal ulcers.

A subject is “susceptible to developing an NSAID-induced gastric and/or duodenal ulcer” if the subject in more susceptible than the average individual to develop an ulcer when under treatment with an NSAID. A high odds ratio for risk of development of NSAID-associated ulcer complications is seen in individuals with a past complicated ulcer (odds ratio 13.5), individuals taking multiple NSAIDs or NSAIDs plus aspirin (odds ratio 9.0); individuals taking high doses of NSAIDs (odds ratio 7.0), individuals under anticoagulant therapy, such as low dose aspirin (odds ration 6.4), individuals with a past uncomplicated ulcer (odds ratio 6.1), and individuals older than 70 years (odds ratio 5.6) See, e.g., Gabriel et al., 1991, Ann Intern Med. 115:787: Garcia Rodriguez et al. 1994, Lancet 343:769; Silverstein et al. 1995, Ann Intern Med. 123:241; and Sorensen et al., 2000, Am J Gastroenterol. 95:2218. Subjects susceptible to developing an NSAID-induced ulcer may have one or more of these risk factors. Subjects “at high risk for developing an NSAID-induced ulcer” are individuals older than 80 years of age and subjects with a history of NSAID-associated serious gastrointestinal complications (perforation of ulcers, gastric outlet obstruction due to ulcers, gastrointestinal bleeding).

### EXAMPLES

### Example 1

### Pharmaceutical Unit Dosage Form I

An exemplary pharmaceutical unit dosage form in accordance with certain embodiments of the invention may be prepared as follows.

A controlled release tablet including 26.6 mg of famotidine and HPMC is prepared via wet granulation techniques, as recognized by those skilled in the art. The tablet is prepared with dimensions suitable for inclusion within a standard pharmaceutical capsule, and with sufficient HPMC such that linear, zero-order release of famotidine is observed under in-vitro assay conditions for at least 4 hours.

A flowable powder including 250 or 500 mg of naproxen (or a corresponding amount of a suitable salt) and a lactose monohydrate bulking agent to balance of 1000 mgs is prepared. If needed, about 5-6 mg of magnesium stearate lubricant may be included in the powder to facilitate processing (with an adjustment in the bulking agent to balance of 1000 mgs).

The tablet and the flowable powder are then combined in a capsule to form a final pharmaceutical unit dosage form in accordance with certain embodiments of the invention.

All literature and patent citations above are hereby expressly incorporated by reference at the locations of their citation. Specifically cited sections or pages of the above cited works are incorporated by reference with specificity. The invention has been described in detail sufficient to allow one of ordinary skill in the art to make and use the subject matter of the following claims. It is apparent that certain modifications of the methods and compositions of the following claims can be made within the scope and spirit of the invention.

What is claimed is:

1. A pharmaceutical unit dosage form, comprising: (a) a first component comprising an amount of an H₂-receptor antagonist effective to raise gastric pH above about 3.5, wherein the first component is formulated so as to allow for sustained release of the H₂-receptor antagonist at said effective amount over a predetermined period of time; and (b) a second component comprising a therapeutically effective amount of an NSAID, wherein the second component is formulated to allow for immediate release of the NSAID.

2. The pharmaceutical unit dosage form of claim 1, wherein the first component is formulated so as to allow for sustained release of the H₂-receptor antagonist at said effective amount for at least about 4 hours.

3. The pharmaceutical unit dosage form of claim 1, wherein the first component is formulated so as to allow for sustained release of the H₂-receptor antagonist at said effective amount for at least about 8 hours.

4. The pharmaceutical unit dosage form of claim 1, wherein the dosage form is a capsule.

5. The pharmaceutical unit dosage form of claim 1, wherein the first component comprises a release modifying agent to, at least in part, provide for said sustained release.

6. The pharmaceutical unit dosage form of claim 5, wherein said release modifying agent is a polymer selected from the group consisting of cellulosic materials, polyvinyl acetates, polyvinyl alcohols, polyethylene oxides, polyethylene glycols, metacrylates, non-crosslinked polyvinylpyrolidone, and combinations thereof.

7. The pharmaceutical unit dosage form of claim 6, wherein the release modifying agent is hydroxypropylmethyl cellulose.

8. The pharmaceutical unit dosage form of claim 1, wherein the first component is formulated as a tablet within the pharmaceutical unit dosage form.

9. The pharmaceutical unit dosage form of claim 1, wherein said H₂-receptor antagonist consists essentially of famotidine or a pharmaceutically acceptable salt thereof.

10. The pharmaceutical unit dosage form of claim 9, comprising from about 20 to about 60 mg of famotidine, or a pharmaceutically acceptable salt thereof.

11. The pharmaceutical unit dosage form of claim 9, comprising about 20 to about 30 mg of famotidine, or a pharmaceutically acceptable salt thereof.

12. The pharmaceutical unit dosage form of claim 1, wherein the second component comprises one or more pharmaceutical acceptable excipients selected from the group consisting of sugars, soluble salts, colorants, fillers, disintegrants, glidants, anti-lacking agents, anti-static agents, and combinations thereof.

13. The pharmaceutical unit dosage form of claim 12, wherein said pharmaceutical acceptable excipients are selected from the group consisting of colloidal silica, calcium diphosphate, talc, magnesium stearate, and combinations thereof.

14. The pharmaceutical unit dosage form of claim 1, wherein said NSAID consists essentially of naproxen, or a pharmaceutically acceptably salt thereof.

15. The pharmaceutical unit dosage form of claim 14, comprising from about 200 to about 600 mg of naproxen, or a pharmaceutically acceptable salt thereof.

16. The pharmaceutical unit dosage form of claim 15, comprising from about 250 to about 500 mg of naproxen, or a pharmaceutically acceptable salt thereof.

17. A pharmaceutical unit dosage form, comprising: (a) a first sustained release component comprising an amount of an H₂-receptor antagonist effective to raise gastric pH above about 3.5 for at least 4 hours, and at least one release modifying agent; and (b) a second immediate release component comprising a therapeutically effective amount of an NSAID; wherein the first sustained release component is formulated as a tablet and the second immediate release component is formulated as a flowable powder; and wherein the pharmaceutical unit dosage form is a capsule comprising said tablet and flowable powder.

18. The pharmaceutical unit dosage form of claim 17, wherein the first sustained release component is effective to raise gastric pH above about 3.5 for at least 8 hours.

19. The pharmaceutical unit dosage form of claim 17, wherein said H₂-receptor antagonist consists essentially of famotidine or a pharmaceutically acceptable salt thereof; said NSAID consists essentially of naproxen, or a pharmaceutically acceptably salt thereof; and said at least one release modifying agent is a polymer selected from the group consisting of: cellulosic materials, polyvinyl acetates, polyvinyl alcohols, polyethylene oxides, polyethylene glycols, metacrylates, non-crosslinked polyvinylpyrolidone, and combinations thereof.

20. A pharmaceutical unit dosage form comprising: (a) a sustained release component comprising from about 20 to about 60 mg of famotidine, or a pharmaceutically acceptable salt thereof, and at least one release modifying agent; and (b) an immediate release component comprising from about 200 to about 600 mg of naproxen, or a pharmaceutically acceptable salt thereof.

21. The pharmaceutical unit dosage form of claim 20, wherein said sustained release component comprises from about 20 to about 30 mg of famotidine, or a pharmaceutically acceptable salt thereof.

22. The pharmaceutical unit dosage form of claim 20, wherein said immediate release component comprises from about 200 to about 500 mg of naproxen, or a pharmaceutically acceptable salt thereof.

23. The pharmaceutical unit dosage form of claim 20, wherein the at least one release modifying agent is a polymer selected from the group consisting of: cellulosic materials, polyvinyl acetates, polyvinyl alcohols, polyethylene oxides, polyethylene glycols, metacrylates, non-crosslinked polyvinylpyrolidone, and combinations thereof.

24. A method for treating osteoarthritis in a subject susceptible to developing NSAID induced gastric and duodenal ulcers, the method comprising administering a pharmaceutical unit dosage form, comprising: (a) a first component comprising an amount of an H₂-receptor antagonist effective to raise gastric pH above about 3.5, wherein the first component is formulated so as to allow for sustained release of the H₂-receptor antagonist at said effective amount over a predetermined period of time; and (b) a second component comprising a therapeutically effective amount of an NSAID, wherein the second component is formulated to allow for immediate release of the NSAID; to a subject in need thereof.

25. A method for treating or preventing pain or inflammation in a subject susceptible to developing NSAID induced gastric and duodenal ulcers, the method comprising administering a pharmaceutical unit dosage form, comprising: (a) a first component comprising an amount of an H₂-receptor antagonist effective to raise gastric pH above about 3.5, wherein the first component is formulated so as to allow for sustained release of the H₂-receptor antagonist at said effective amount over a predetermined period of time; and (b) a second component comprising a therapeutically effective amount of an NSAID, wherein the second component is formulated to allow for immediate release of the NSAID; to a subject in need thereof.

26. A method for preparing a pharmaceutical unit dosage form, the method comprising:
(a) preparing a first sustained release matrix core comprising an amount of an H₂-receptor antagonist effective to raise gastric pH above about 3.5 over a predetermined period of time following administration and at least one release modifying agent;
(b) preparing an immediate release component comprising a therapeutically effective amount of an NSAID; and
(c) combining the matrix core of step (a) and the immediate release blend of step (b) within a unit dosage form.

27. The method as defined in claim 26, wherein the release modifying agents comprises one or more polymers.

28. The method as defined in claim 27, wherein said polymers are selected from the group consisting of cellulosic materials, polyvinyl acetates, polyvinyl alcohols, polyethylene oxides, polyethylene glycols, methacrylates, non-crosslinked polyvinylpyrolidone, and combinations thereof.

29. The method as defined in claim 26, wherein said immediate release component further comprises one or more pharmaceutically acceptable excipients selected from the group consisting of cellulose derivatives, cross-linked polymers, sugars, soluble salts, colorants, fillers, disintegrants, glidants, anti-tacking agents and anti-static agents.

30. The method as defined in claim 29, wherein said pharmaceutical acceptable excipients are selected from the group consisting of colloidal silica, calcium diphosphate, talc, magnesium stearate, and combinations thereof.

31. The method as defined in claim 26, wherein the sustained release matrix is formulated as a tablet.

32. The method as defined in claim 26, wherein the immediate release component is formulated as a powder blend.

33. The method as defined in claim 26, wherein the pharmaceutical unit dose is a capsule, and the method further comprises compressing the sustained release matrix into the form of a tablet; forming the immediate release component as a flowable powder; and loading the sustained release tablet and flowable immediate release powder into the capsule to form the pharmaceutical unit dose.

34. The method as defined in claim 26, wherein the H₂-receptor antagonist consists essentially of famotidine, or a pharmaceutically acceptable salt thereof.

35. The method as defined in claim 26, wherein the NSAID consists essentially of naproxen, or a pharmaceutically acceptably salt thereof.
